(19) United States
(12) Patent Application Publication
(10) Pub. No.: US 2017/0112518 A1
(43) Pub. Date: Apr. 27, 2017

(54) CANNULATION DEVICES

(71) Applicant: Mayo Foundation for Medical Education and Research, Rochester, MN (US)

(72) Inventor: Mark D. Topazian, Rochester, MN (US)

(73) Assignee: Mayo Foundation for Medical Education and Research, Rochester, MN (US)

(21) Appl. No.: 15/315,513

(22) PCT Filed: Jun. 5, 2015
(86) PCT No.: PCT/US15/34391

§ 371 Date: Dec. 1, 2016

Related U.S. Application Data
(60) Provisional application No. 62/008,010, filed on Jun. 5, 2014.

(51) Int. Cl. A61B 17/29; A61B 17/00; A61B 17/30; A61M 25/00; A61B 17/34

(52) U.S. Cl. CPC A61B 17/29; A61M 25/0082; A61B 17/3478; A61B 17/30; A61B 17/00234; A61B 2017/2906; A61B 2017/308; A61B 2017/3488; A61B 2017/00292; A61B 2017/00296; A61B 2017/00818

(57) ABSTRACT

Devices and methods are provided for facilitating canulation of body conduits. For example, this document provides devices and methods for facilitating canulation of the bile and/or pancreatic ducts in the context of an endoscopic retrograde cholangiopancreatography (ERCP) procedure.

### CROSS REFERENCE TO RELATED APPLICATIONS

This application claims the benefit of U.S. Provisional Ser. No. 62/008,010 filed Jun. 5, 2014. The disclosure of the prior application is considered part of (and is incorporated by reference in) the disclosure of this application.

### BACKGROUND

1. Technical Field

This document relates to devices and methods for facilitating canulation of body conduits. For example, this document relates to devices and methods for facilitating canulation of the bile and/or pancreatic ducts in the context of an endoscopic retrograde cholangiopancreatography procedure.

2. Background Information

Endoscopic retrograde cholangiopancreatography (ERCP) is an endoscopic technique for examining and treating problems in the bile and pancreatic ducts, and the gallbladder. For example, ERCP can be used to identify and treat stones, tumors, or narrowing in the bile and pancreatic ducts. The bile ducts are channels that drain bile from the liver and gallbladder. The pancreatic ducts are channels that drain the pancreas. Both types of ducts empty into the duodenum, which is the first part of the small intestine.

ERCP is performed by gastroenterologists or surgeons using an endoscope, which is a long, flexible tube with a camera and a light at the tip. First, the patient is sedated to the point of appearing asleep. The endoscope or tube is placed into the patient's mouth and advanced into the esophagus, stomach, and to the point in the duodenum where the bile duct enters the duodenum. The point where the bile ducts enters the duodenum is known as the papilla of Vater (or simply the papilla). A plastic catheter (thin tube) or a guidewire is threaded through the scope so as to extend from the distal tip portion of the scope. The catheter or guidewire is then maneuvered into position to enter the papilla and the ducts. In some cases, contrast dye (radiopaque material) is injected from the catheter into the ducts and x-rays are taken to acquire information about blockages, stones, tumors, or irregularities of the ducts. If abnormalities are found, the doctor can perform techniques to repair or improve the condition. For example, if stones are seen, the papilla can be widened or cut open and stones may be removed from the duct. In some cases, the guidewire can be used to assist with the placement of one or more interventional stent devices that can facilitate patency of the ducts.

### SUMMARY

This document provides devices and methods for facilitating canulation of body conduits. For example, this document provides devices and methods for facilitating canulation of the bile and/or pancreatic ducts in the context of an endoscopic retrograde cholangiopancreatography procedure.

In one implementation, a device for performing cannulation includes an elongate catheter, the catheter defining a lumen therethrough; and a tissue stabilization device movably coupled to the catheter.

Such a device for performing cannulation may optionally include one or more of the following features. The tissue stabilization device may comprise a grasping device. The tissue stabilization device may further comprise a wire that is coupled to the tissue stabilization device and to the catheter such that the orientation of the tissue stabilization device and the catheter can be adjusted by extending or retracting the wire. The device may further comprising a wire that is coupled to the tissue stabilization device such that the orientation of the tissue stabilization device in relation to the catheter can be adjusted by extending or retracting the wire. The tissue stabilization device may comprise two or more grasping devices. The individual grasping devices of the two or more grasping devices can be actuated independently of other individual grasping devices of the two or more grasping devices. The device may further comprise an elongate sheath, wherein the sheath is slidably disposed over the catheter, and wherein the sheath defines two or more lumens in which the two or more grasping devices are disposed. The tissue stabilization device may comprise a barbed component pivotably disposed on the catheter. The device may further comprise an actuator configured to expand or retract the barbed component. The tissue stabilization device may comprise a suction device. The tissue stabilization device may comprise two or more suction devices. Optionally, the individual suction devices of the two or more suction devices can be actuated independently of other individual suction devices of the two or more suction devices. The device may further comprise an elongate sheath, wherein the sheath is slidably disposed over the catheter, and wherein the sheath defines two or more lumens in which the two or more suction devices are disposed.

In another implementation, a system for performing cannulation includes an endoscope with a working channel; an elongate catheter slidably disposable within the working channel, the catheter defining a lumen therethrough; and a tissue stabilization device movably coupled to the catheter.

Such a system for performing cannulation may optionally include one or more of the following features. The tissue stabilization device may comprise a grasping device. The tissue stabilization device may comprise two or more grasping devices. The system may further comprise an elongate sheath, wherein the sheath is slidably disposed over the catheter, and wherein the sheath defines two or more lumens in which the two or more grasping devices are disposed. The tissue stabilization device may comprise a barbed component pivotably disposed on the catheter. The tissue stabilization device may comprise a suction device. The tissue stabilization device may comprise two or more suction devices. Optionally, the individual suction devices of the two or more suction devices can be actuated independently of other individual suction devices of the two or more suction devices.

In another implementation, a method for cannulation includes navigating an insertion tube of an endoscope to a target site within a patient; extending a catheter from a distal end portion of the insertion tube; extending a tissue stabilization device movably coupled to the catheter from the distal end portion of the insertion tube; manipulating, using the tissue stabilization device, tissue of the patient; and inserting the catheter into an orifice of the patient.

Such a method for cannulation may optionally include one or more of the following features. The orifice may be a papilla of Vater. The tissue stabilization device may be a grasping device. The tissue stabilization device may be a suction device.

Particular embodiments of the subject matter described in this document can be implemented to realize one or more of the following advantages. In some embodiments, the devices and methods provided herein can facilitate improvements in the canulation of body conduits. For example, in some cases a patient's point of canulation (e.g., the papilla or other orifice/ostium) is difficult to visualize or access. In some such cases, the devices and methods provided herein can be used to manipulate the tissue surrounding the point of canulation to thereby improve the visualization and/or access. Consequently, some procedures that may otherwise require open surgery can be performed using minimally invasive endoscopic or transcatheter techniques. Patient discomfort, recovery times, treatment costs, and risks may thereby be abated in some cases.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1A is a schematic diagram of patient undergoing an ERCP in accordance with some embodiments provided herein.

FIG. 1B is an enlarged partial cutaway view illustrating the canulation of the patient's papilla.

FIG. 2A is a partial cutaway view illustrating an in-process ERCP prior to canulation.

FIG. 2B is an enlarged view of an example device that can be used to facilitate canulation of the patient's papilla.

FIGS. 3A and 3B are enlarged views of another example device that can be used to facilitate canulation of the patient's papilla.

FIG. 4 is an enlarged view of another example device that can be used to facilitate canulation of the patient's papilla.

FIG. 5 is an enlarged view of another example device that can be used to facilitate canulation of the patient's papilla.

FIG. 6 is an enlarged view of another example device that can be used to facilitate canulation of the patient's papilla.

FIG. 7 is an enlarged view of another example device that can be used to facilitate canulation of the patient's papilla.

FIG. 8 is an enlarged view of another example device that can be used to facilitate canulation of the patient's papilla.

FIG. 9 is an enlarged view of another example device that can be used to facilitate canulation of the patient's papilla.

Like reference numbers represent corresponding parts throughout.

### DETAILED DESCRIPTION

This document provides devices and methods to improve cannulation of body conduits including, but not limited to, the biliary duct or pancreatic duct at the papilla of Vater during ERCP. While cannulation of the papilla during ERCP is used as the context to describe the devices and methods provided herein, it should be understood that the devices and methods may also be used in various other suitable contexts. For example, devices and methods provided herein may be used to cannulate fistulas or strictures in any body conduit.

In a general sense, the devices provided herein are used to grasp, stabilize, or tether the tissue surrounding the point of canulation (e.g., papilla or other orifice/ostium), enabling the clinician to draw the papilla towards the duodenoscope or otherwise move the papilla in various directions. Such manipulative movements can be used to enhance endoscopic visualization and access to the papilla, and to straighten or stabilize the position of the intra-ampullary ducts so that they can be cannulated more easily.

In some embodiments, the devices include one or more grasping jaws near the tip of a cannula, sphincterotome, or other ERCP accessory. The jaw(s) can be used to grasp the lip of the ampullary orifice or other nearby tissue, and the device can then be used to manipulate the tissue to a desired orientation. Thereafter, the desired orifice and duct can be cannula with a guidewire, a cannula, or other device. In some embodiments, other means of grasping or tethering the papilla may be incorporated into the device, such as a tripod-style grasper with two or more arms, barbs or flanges on the cannula, threads on the cannula, one or more suction devices, and the like. Such features may be included at the tip portion on the shaft of a cannula or accessory device.

It should be understood that features from one device embodiment provided herein can be combined with one or more features of one or more other device embodiments provided herein, without limitation. To provide an example, a grasping tool from one embodiment can be combined with a threaded or barbed cannula from another embodiment. Moreover, all features provided herein (and equivalents thereof) can be mixed and matched to create hybrid device designs, and such device designs are within the scope of this disclosure.

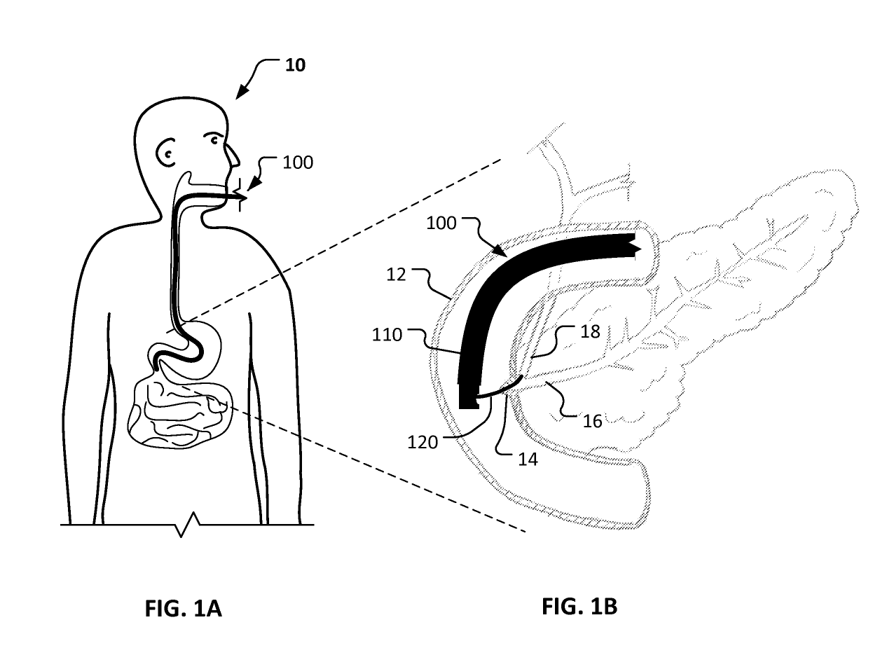

Referring to FIGS. 1A and 1B, a patient 10 can undergo ERCP using a duodenoscope system 100. Duodenoscope system 100 includes an elongate flexible insertion tube 110 that can be navigated by a clinician operator to a target position within the duodenum 12 of patient 10.

In some embodiments, insertion tube 100 includes one or more lumens or channels therethrough. Such lumens may be used for purposes such as, but not limited to: providing irrigation fluid; providing suction; biopsy containment; fiber optic strand(s); camera wires; electrocautery wires; ultrasound wires; control wires for steering or manipulating insertion tube 100, end effect devices, and the like; working channels for one or more catheters, guidewires, grasping devices, suction devices, and the like; conveyance of contrast media; and the like.

In the depicted implementation, insertion tube 110 has at least a catheter 120 slidably disposed therein. Catheter 120 can be used to cannulate a papilla of Vater 14 of patient 10. The cannulation process can be used to access a pancreatic duct 16 or a common bile duct 18 of patient 10, for example. In some cases, a contrast media may be injected via catheter 14 to allow for fluoroscopic visualization of pancreatic duct 16 or common bile duct 18. In some cases, another type of device, such as a guidewire, may be used to perform the cannulation rather than catheter 120.

In some circumstances, papilla 14 can be quite difficult to cannulate. For example, in some cases a patient's papilla 14 may be located in a diverticulum that can make visualization and access to papilla 14 particularly challenging. In some cases, a patient's papilla 14 may be small and/or flat on the wall of duodenum 12, and therefore difficult to visualize and access for cannulation. In some cases, the patient's papilla 14 may be stenotic or the ampullary vestibule may be overly capacious. Further, the intra-ampullary ducts may be sharply angulated preventing deep cannulation in some cases. These types of situations, and others, can tend to make cannulation of the papilla 14 difficult to achieve.

While the cannulation devices provided herein are described in the context of an endoscopic delivery system, it should be understood that the endoscope is not required in all implementations of the cannulation devices. For example, in some embodiments a delivery sheath can be used to deliver the cannulation devices to a target site within patient 10. In some embodiments, x-ray fluoroscopy (or another imaging modality) can be used in conjunction with the devices and systems provided herein. Therefore, in some embodiments radiopaque markers or echogenic regions are included on some portions of the devices provided herein.

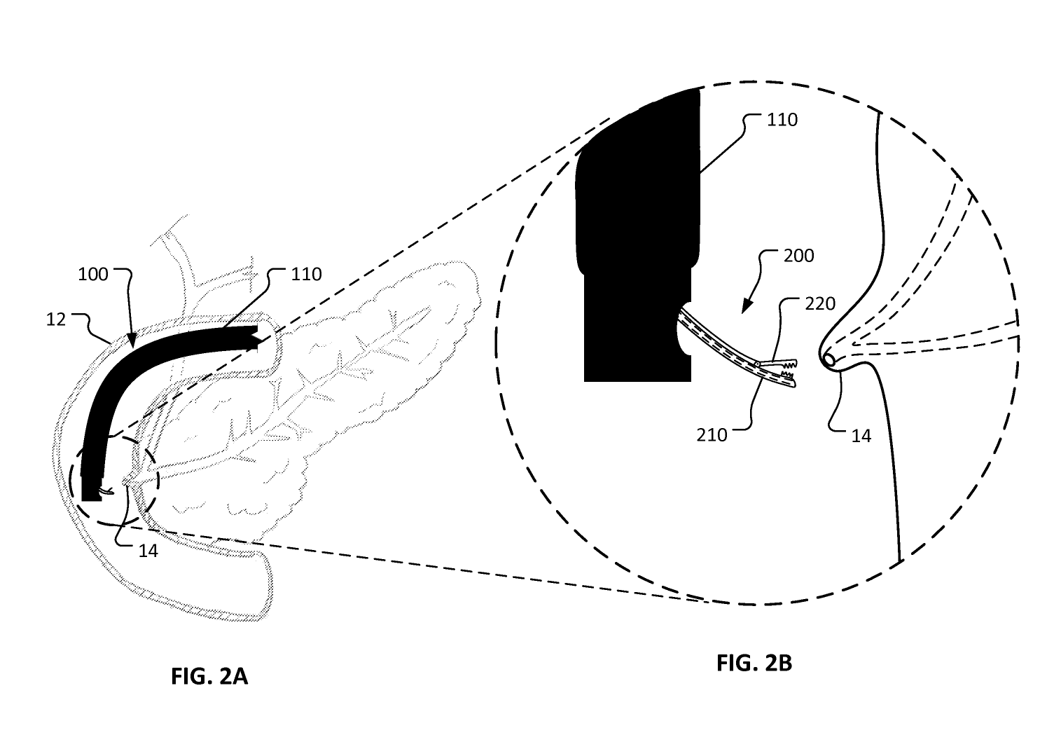

Referring to FIGS. 2A and 2B, endoscopic insertion tube 110 can be used to deliver an example cannulation device 200 to the target site of papilla 14 within duodenum 12. Cannulation device 200 can be slidably disposed within one or more lumens of insertion tube 110. Accordingly, multiple degrees of freedom are available to the clinician operator to manipulate the position and orientation of cannulation device 200. For example, in some embodiments at least the distal end portion of insertion tube 110 is steerable so as to angulate the distal end portion (and cannulation device 200) in relation to papilla 14.

In the depicted embodiment, cannulation device 200 includes a catheter 210 and a grasping device 220. Catheter 210 includes at least one lumen through which a clinician can inject materials (e.g., contrast media, saline, a guidewire, and the like) or provide suction for aspiration, for example. Grasping device 220 includes one or more sets of pivotably actuatable forceps or jaws. Such forceps or jaws can be used, for example, to grasp and manipulate the lip of papilla 14 or other tissue. The clamping action of grasping device 220 can be activated and deactivated by the clinician operator.

In some embodiments, catheter 210 and grasping device 220 are conjoined, and are therefore configured to be extended and retracted in a consolidated fashion. In some embodiments, catheter 210 and grasping device 220 can be manipulated separately or at least partially independently of each other. For example, in some embodiments, catheter 210 and grasping device 220 can be extended and retracted independently of each other, while remaining laterally tethered adjacent to each other. In some embodiments, catheter 210 and grasping device 220 are not attached to each other at all.

Grasping device 220 can be operated to grasp and manipulate the lip of papilla 14 or other tissue, including tissue surrounding papilla 14. Concurrently, the clinician operator can visualize the region using the endoscopic insertion tube 110. When the papilla 14 is thereby orientated in a desired position by operation of grasping device 220, catheter 210 can be further extended so as to insert it through papilla 14.

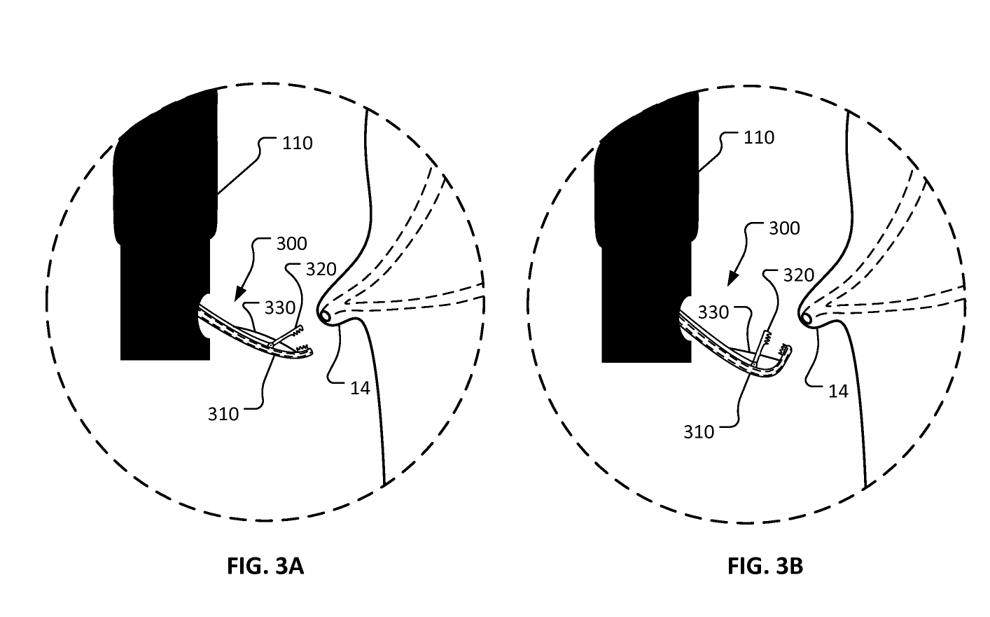

Referring to FIGS. 3A and 3B, endoscopic insertion tube 110 can be used to deliver another example cannulation device 300 to the target site of papilla 14. Cannulation device 300 includes a catheter 310, a grasping device 320, and a wire 330. Catheter 310 and grasping device 320 can be configured with the features and operated in the manner essentially like catheter 210 and grasping device 220 described above.

Wire 330 can be extended and retracted by a clinician operator. FIG. 3A exemplifies the configuration of cannulation device 300 when wire 330 is in the extended, or essentially extended, configuration. FIG. 3B, exemplifies the configuration of cannulation device 300 when wire 330 is in the retracted, or essentially retracted, configuration. It can be seen that retraction of wire 330 can reorient a distal end portion of catheter 310 and grasping device 320 (refer to FIG. 3B). In some embodiments, wire 330 reorients just one of either catheter 310 or grasping device 320. In some embodiments, wire 330 reorients both of catheter 310 and grasping device 320.

In some embodiments, wire 330 can be a metallic wire (e.g., nitinol or stainless steel) or another suitable material. As such, wire 330 can have a column strength sufficient to forcibly extend catheter 310 and/or grasping device 320. In some embodiments, catheter 310 and/or grasping device 320 are biased to be in an extended configuration (e.g., refer to FIG. 3A) so that wire 330 need not forcibly extend catheter 310 and/or grasping device 320. In some such embodiments, wire 330 can be made of suture material, very thin metallic wire, or other suitable materials. In some embodiments, wire 330 can be an electrocautery wire.

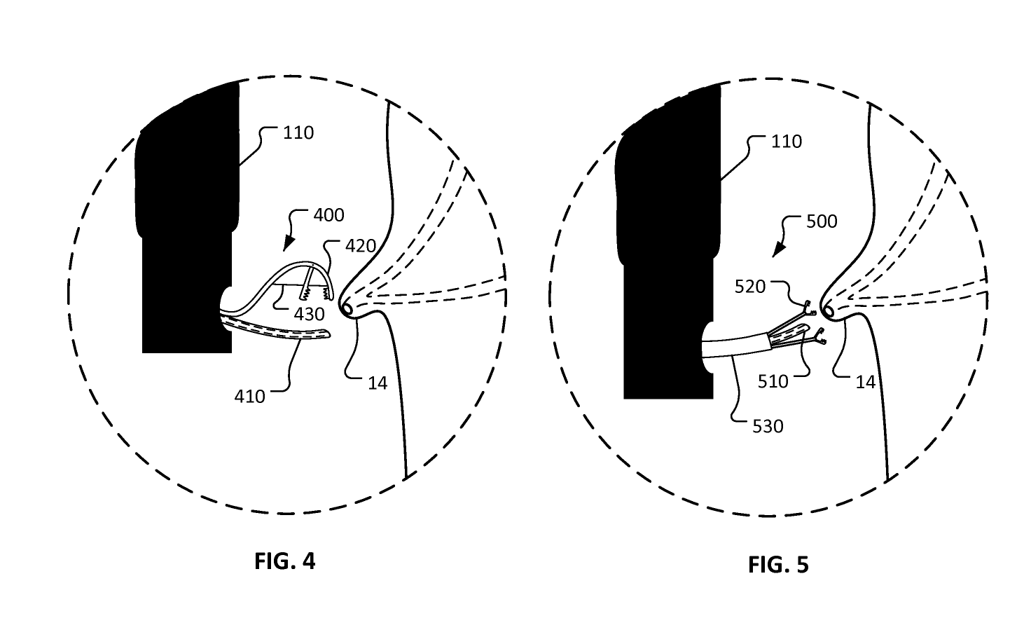

Referring to FIG. 4, endoscopic insertion tube 110 can be used to deliver another example cannulation device 400 to the target site of papilla 14. Cannulation device 400 includes a catheter 410, a grasping device 420, and an optional wire 430. Catheter 410 and grasping device 420 can be configured with the features and operated in the manner essentially like catheter 210 and grasping device 220 described above. However, in the depicted embodiment the grasping device 220 is specifically configured to approach papilla 14 at a separate angle from catheter 410.

Grasping device 420 can be operated to grasp and manipulate the lip of papilla 14 or other tissue, including tissue surrounding papilla 14. Concurrently, the clinician operator can visualize the region using the endoscopic insertion tube 110. When the papilla 14 is thereby orientated in a desired position by operation of grasping device 420, catheter 410 can be inserted through papilla 14.

Grasping device 420 can optionally include wire 430. Wire 430 can be configured similarly to wire 330 described above. That is, wire 430 can be retracted or extended to reorient the distal end portion of grasping device 420 as desired by the clinician operator. FIG. 4 depicts wire 430 in a retracted configuration. In some embodiments, catheter 410 can also be configured with a wire that can be extended and retracted to reorient a distal end portion of catheter 410 as desired by the clinician operator.

Referring to FIG. 5, endoscopic insertion tube 110 can be used to deliver another example cannulation device 500 to the target site of papilla 14. Cannulation device 500 includes a catheter 510, one or more grasping devices 520, and an optional sheath 530. Catheter 510 and grasping devices 520 can be configured with the features and operated in the manner essentially like catheter 210 and grasping device 220 described above. However, in the depicted embodiment two grasping devices 520 are included. In some embodiments, three, four, or more than four grasping devices 520 can be included.

In some embodiments, each grasping device of the grasping devices 520 can be operated independently. That is each grasping device of the grasping devices 520 can be extended, retracted, opened, closed, and otherwise manipulated essentially independently of any other grasping device of the grasping devices 520. In some embodiments, one or more aspects of the grasping devices 520 can be operated in concert with each other.

In some embodiments, optional sheath 530 is slidably disposed over catheter 510. Sheath 530 can include one or more lumens in which grasping devices 520 can be slidably disposed. Sheath 530 can advantageously facilitate predictable orientation and operation of grasping devices 520 in some circumstances.

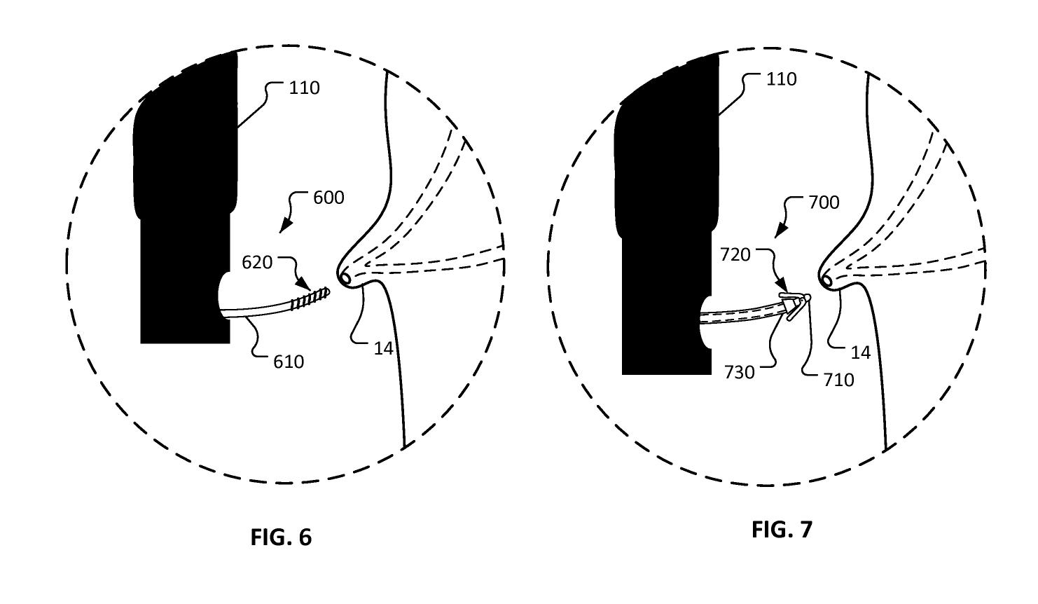

Referring to FIG. 6, endoscopic insertion tube 110 can be used to deliver another example cannulation device 600 to the target site of papilla 14. Cannulation device 600 includes a catheter 610 having a threaded portion 620. Catheter 610 is rotatable, by the clinician operator, in relation to endoscopic insertion tube 110 and to papilla 114. As catheter 610 is rotated, threaded portion 620 may seek to essentially engage and advance into and/or through papilla 14, that is, to cannulate papilla 14. In that fashion, threaded portion 620 may assist with the cannulation of papilla 14.

Referring to FIG. 7, endoscopic insertion tube 110 can be used to deliver another example cannulation device 700 to the target site of papilla 14. Cannulation device 700 includes a catheter 710, a barb component 720, and an optional wedge actuator 730. In this embodiment, barb component 720 is configured to grasp or engage papilla 14 upon insertion of the distal tip portion of catheter 710 through papilla 14.

In some embodiments, barb component 720 is configured to be pivotably expanded or retracted using wedge actuator 730. Wedge actuator 730 can comprise a tube that is slidably disposed over catheter 710, and a wedge-shaped end effector can be disposed on the distal end of the tube. The clinician operator can extend or retract wedge actuator 730 to cause the expansion or retraction of barb component 720.

In some embodiments, barb component 720 is naturally biased to be in the retracted configuration. Therefore, when wedge actuator 730 is retracted, barb component 720 will naturally retract towards the outer diameter of catheter 710.

After placement of the distal tip portion of catheter 710 through papilla 14, the clinician operator can extend wedge actuator 730 to expand barb component 720. In that configuration, cannulation device 700 can straighten or stabilize the position of the intra-ampullary ducts, and be retained in such a configuration by the forces exerted by the barb component 720 on the duct walls. Thereafter, to retract the cannulation device 700, the clinician operator can retract wedge actuator 730 to allow barb component 720 to retract towards the outer diameter of catheter 710. The catheter can be retracted from papilla 14. While this embodiment uses pivotable barbs, in some embodiments other types of protrusions (e.g., coils, needles, and the like) can be used to achieve a similar affect.

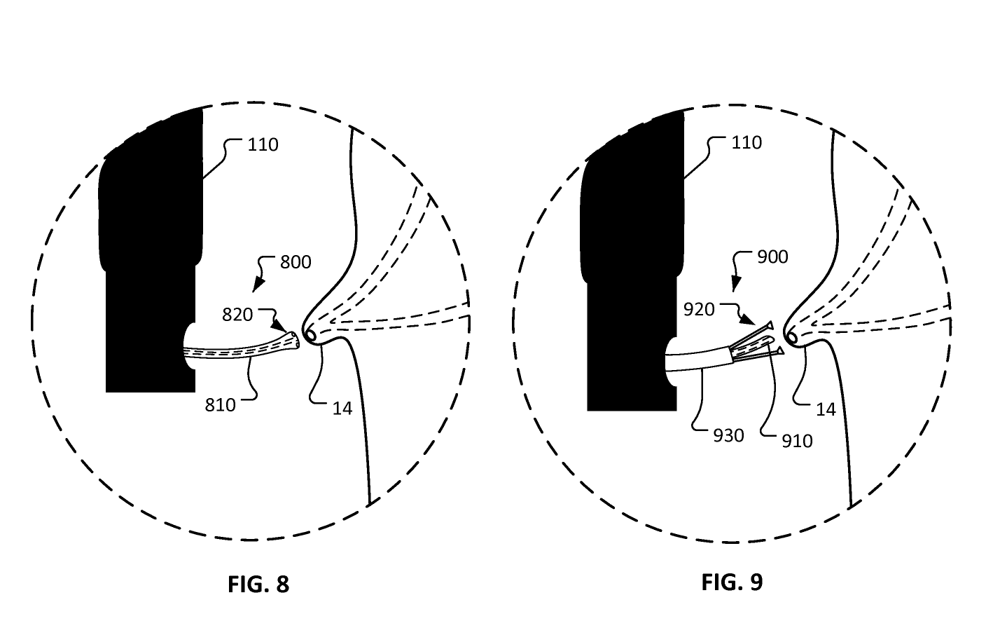

Referring to FIG. 8, endoscopic insertion tube 110 can be used to deliver another example cannulation device 800 to the target site of papilla 14. Cannulation device 800 includes a catheter 810 having a distal suction portion 820. Distal suction portion 820 includes one or more suction orifices at or around the distal tip portion of catheter 810, such as around the outlet of the main lumen of catheter 810. Distal suction portion 820 can be used to releasably attach catheter 810 to the tissue around papilla 14, to thereby stabilize catheter 810 relative to papilla 14. Such stabilization can serve to facilitate cannulation of papilla 14 by catheter 810 in some circumstances.

Referring to FIG. 9, endoscopic insertion tube 110 can be used to deliver another example cannulation device 900 to the target site of papilla 14. Cannulation device 900 includes a catheter 910, one or more suction devices 920, and an optional sheath 930. In some embodiments, catheter 910 can be extended and/or retracted independently from the extension and retraction of suction devices 920.

Cannulation device 900 can include one, two, three, four, or more than four individual suction members to comprise the suction devices 920. In some embodiments, the clinician operator can independently extend and/or retract each individual suction device of the suction devices 920 in relation to the other suction device(s) of the suction devices 920. In some embodiments, the clinician operator can extend and/or retract each suction device of the suction devices 920 in unison.

Suction devices can be used to releasably attach cannulation device 900 to the tissue around papilla 14 to thereby stabilize catheter 910 relative to papilla 14. Such stabilization can serve to facilitate cannulation of papilla 14 by catheter 910 in some circumstances.

Alternatively, or additionally, an elongated, pyramidal, or peanut-shaped balloon near or at the tip of a catheter that can be inflated within the ampullary orifice to stabilize the papilla for cannulation is also envisioned within the scope of this disclosure. In some such embodiments, ridges or flanges are included on the surface of the balloon. The balloons can be pushed into tissue when the balloon is inflated, thereby grasping and stabilizing the ampulla.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A device for performing cannulation, the device comprising:
an elongate catheter, the catheter defining a lumen therethrough; and
a tissue stabilization device movably coupled to the catheter.

2. The device of claim 1, wherein the tissue stabilization device comprises a grasping device.

3. The device of claim 1, further comprising a wire that is coupled to the tissue stabilization device and to the catheter such that the orientation of the tissue stabilization device and the catheter can be adjusted by extending or retracting the wire.

4. The device of claim 3, further comprising a wire that is coupled to the tissue stabilization device such that the orientation of the tissue stabilization device in relation to the catheter can be adjusted by extending or retracting the wire.

5. The device of claim 1, wherein the tissue stabilization device comprises two or more grasping devices and wherein individual grasping devices of the two or more grasping devices can be actuated independently of other individual grasping devices of the two or more grasping devices.

6. (canceled)

7. The device of claim 6, further comprising an elongate sheath, wherein the sheath is slidably disposed over the catheter, and wherein the sheath defines two or more lumens in which the two or more grasping devices are disposed.

8. The device of claim 1, wherein the tissue stabilization device comprises a barbed component pivotably disposed on the catheter, and wherein the device further comprises an actuator configured to expand or retract the barbed component.

9. (canceled)

10. The device of claim 1, wherein the tissue stabilization device comprises a suction device.

11. The device of claim 1, wherein the tissue stabilization device comprises two or more suction devices, and wherein individual suction devices of the two or more suction devices can be actuated independently of other individual suction devices of the two or more suction devices.

12. (canceled)

13. The device of claim 12, further comprising an elongate sheath, wherein the sheath is slidably disposed over the catheter, and wherein the sheath defines two or more lumens in which the two or more suction devices are disposed.

14. A system for performing cannulation, the system comprising:
an endoscope with a working channel;
an elongate catheter slidably disposable within the working channel, the catheter defining a lumen therethrough; and
a tissue stabilization device movably coupled to the catheter.

15. The system of claim 14, wherein the tissue stabilization device comprises a grasping device.

16. The system of claim 14, wherein the tissue stabilization device comprises two or more grasping devices.

17. The system of claim 16, further comprising an elongate sheath, wherein the sheath is slidably disposed over the catheter, and wherein the sheath defines two or more lumens in which the two or more grasping devices are disposed.

18. The system of claim 14, wherein the tissue stabilization device comprises a barbed component pivotably disposed on the catheter.

19. The system of claim 14, wherein the tissue stabilization device comprises suction device.

20. (canceled)

21. (canceled)

22. A method for cannulation, the method comprising:
navigating an insertion tube of an endoscope to a target site within a patient;
extending a catheter from a distal end portion of the insertion tube;
extending a tissue stabilization device movably coupled to the catheter from the distal end portion of the insertion tube;
manipulating, using the tissue stabilization device, tissue of the patient; and
inserting the catheter into an orifice of the patient.

23. The method of claim 22, wherein the orifice is a papilla of Vater.

24. The method of claim 22, wherein the tissue stabilization device is a grasping device.

25. The method of claim 22, wherein the tissue stabilization device is a suction device.
